(19) United States
(12) Patent Application Publication
(10) Pub. No.: US 2024/0318126 A1
(43) Pub. Date: Sep. 26, 2024

(54) IN-SITE COMPOSTING SYSTEM FOR COMMUNITY KITCHEN WASTE

(71) Applicant: China Agricultural University, Beijing (CN)

(72) Inventors: Yanming LI, Beijing (CN); Ruixue CHANG, Beijing (CN); Jue WANG, Beijing (CN); Rulu QIAO, Beijing (CN); Jie YANG, Beijing (CN); Xiaoqing WU, Beijing (CN); Xin MENG, Beijing (CN); Jiaqi LIU, Beijing (CN)

(21) Appl. No.: 18/603,428

(22) Filed: Mar. 13, 2024

(30) Foreign Application Priority Data
Mar. 21, 2023 (CN) 202310280215.2

(51) Int. Cl. C12M 1/00; B09B 3/38; B09B 3/60; B09B 101/70; C12M 1/06; C12M 3/04

(52) U.S. Cl. CPC C12M 43/08; B09B 3/38; B09B 3/60; C12M 27/02; C12M 27/12; B09B 2101/70

(57) ABSTRACT

An in-site composting system for community kitchen waste is provided, which includes: a dump unit, configured to receive a kitchen waste; a power energy conversion unit, configured to convert a part of energy generated by a fitness equipment or a rocking handle into electrical energy, and output a remaining part of the generated energy directly as power, a biological conversion unit, configured to receive the kitchen waste fed by the dump unit and perform a composting and fermentation processing on the kitchen waste; an energy storage unit, configured to store the electrical energy generated by the power energy conversion unit and the solar panel; and a material outputting and returning unit, configured to be connected to the biological conversion unit, wherein the material outputting and returning unit is configured to output and return a material decomposed by the biological conversion unit.

### CROSS-REFERENCE TO RELATED APPLICATION

This patent application claims the priority of Chinese Patent Application No. 202310280215.2 filed with the China National Intellectual Property Administration on Mar. 21, 2023, and entitled “an in-site compositing system for community kitchen waste”, the entire content of which is incorporated by reference in the present application.

### TECHNICAL FIELD

The present disclosure relates to the field of organic solid waste bioresource technology, and in particular to an in-site composting system for community kitchen waste.

### BACKGROUND

Composting technology is an important optimized way to address the resource utilization of the urban kitchen waste. After the implementation of the waste classification policy, the amount of kitchen waste separated has increased sharply, and the situation of overloading operations of centralized waste disposal facilities in various regions has suddenly emerged. The construction of such facilities will lag behind the objective demands of urban development for a long time. Meanwhile, urban expansion will lead to a further increase in the cost of transportation and disposal of the kitchen waste, thereby aggravating the secondary pollution caused by the kitchen waste. So, the small kitchen waste disposal facilities enabling in-site or nearby use are adopted to alleviate the above-mentioned contradictions and can also flexibly adapt to the relatively dispersed characteristics of communities.

At present. the operation of the in-site disposal system for community kitchen waste mainly relies on the power system to maintain its power consumption, resulting in a significant increase in disposal energy consumption and disposal cost. However, the current community fitness system is relatively independent and lacks matched kinetic energy conversion and energy storage systems to achieve maximum energy utilization within communities.

Therefore, there is an urgent need for a community in-site collection and disposal system that occupies a small scale and can reduce the amount of kitchen waste at the source.

### SUMMARY

In the present disclosure, in order to provide a community in-site collection and disposal system that occupies a small scale and can reduce the amount of kitchen waste at the source, the present disclosure adopts the following technical solutions.

An in-site composting system for community kitchen waste includes:

a dump unit, configured to receive a kitchen waste;

a power energy conversion unit. configured to convert a part of energy generated by a fitness equipment or a rocking handle into electrical energy, and output a remaining part of the generated energy as power;

a biological conversion unit, configured to cooperate with the dump unit and receive the power output by the power energy conversion unit, where the biological conversion unit is configured to receive the kitchen waste fed by the dump unit and perform a composting and fermentation processing on the kitchen waste:

an energy storage unit, configured to be connected to the power energy conversion unit and a solar panel, where the energy storage unit is configured to store the electrical energy generated by the power energy conversion unit and the solar panel: and

a material outputting and returning unit, configured to be connected to the biological conversion unit, where the material outputting and returning unit is configured to output and return a material decomposed by the biological conversion unit.

In the embodiment, the power energy conversion unit includes the fitness equipment, a transmission assembly, and a power generation assembly; the transmission assembly includes a flywheel assembly and a driveshaft set. where a main rotating wheel of the fitness equipment is connected to the flywheel assembly in a transmission manner through a directly connected transmission shaft; the driveshaft set includes a first transmission shaft, a second transmission shaft, and a third transmission shaft.

The flywheel assembly is connected to a first end of the first transmission shaft, and the first transmission shaft and the second transmission shaft are arranged vertically: where a second end of the first transmission shaft is drivingly connected to a non-end part of the second transmission shaft through a transmission component.

A first end of the second transmission shaft is provided with a transmission device, the transmission device is drivingly connected to a first end of the third transmission shaft, and the first end of the third transmission shaft is engaged with an input end of the power generation assembly.

In the embodiment, the transmission device is a gearbox: the power generation assembly includes a generator and a current collecting device, an input end of the gearbox is drivingly connected to the first end of the second transmission shaft; an output end of the gearbox is drivingly connected to the first end of the third transmission shaft. and a second end of the third transmission shaft is drivingly connected to the generator through a coupling: and a brake is arranged on the gearbox: and the current collecting device is configured to collect the electrical energy generated by the generator and transmit the electrical energy to the energy storage unit.

In the embodiment, the dump unit includes a kitchen waste can, a gravity sensing gate. a crushing and removing impurity device. and a feeding pipeline. The gravity sensing gate is arranged at a bottom of the kitchen waste can. The crushing and removing impurity device is arranged at a lower end of the gravity sensing gate, and is configured to remove impurities in the kitchen waste and then crush the kitchen waste.

The feeding pipeline is arranged at a lower end of the crushing and removing impurity device, and is configured to transport the kitchen waste processed by the crushing and removing impurity device to the biological conversion unit.

In the embodiment, the biological conversion unit includes a roller fermentation bin, a micro-negative pressure deodorization device, a gear transmission mechanism, a stirring device, and a driving device: the roller fermentation bin may be rotatably installed on a base.

Front and rear ends of the roller fermentation bin are respectively provided with a front-end sealing bin and a rear-end sealing bin.

The micro-negative pressure deodorization device includes a breather pipe, an induced draft fan, an air outlet pipe, and a deodorization filter element. A first end of the breather pipe is communicated with a top of the rear-end sealing bin, and a second end of the breather pipe is connected to an inlet end of the induced draft fan: an outlet end of the induced draft fan is connected to a first end of the air outlet pipe. and the deodorization filter element is arranged on the air outlet pipe and located at a non-end position of the air outlet pipe; and an end part of a second end of the air outlet pipe is provided with a rain proof hood.

The gear transmission mechanism includes an outer ring gear arranged on an outer wall of the roller fermentation bin and a first gear arranged on a fourth transmission shaft, wherein the first gear meshes with the outer ring gear.

The stirring device includes a stirring shaft and a plurality of blades, wherein the stirring shaft is located inside the roller fermentation bin and is sealed and rotatably connected to the roller fermentation bin: the quantity of the blades is multiple, and the plurality of the blades are arranged on a circumferential outer side of the stirring shaft.

The driving device includes an electric motor arranged on an outer side of the roller fermentation bin, and an output shaft of the electric motor is drivingly connected to the fourth transmission shaft.

A feeding port of the front-end sealing bin is arranged to correspond to the feeding pipeline. and a bottom of the rear-end sealing bin is provided with a discharge port.

In the embodiment, the material outputting and returning unit includes a hopper, a lifting rope, a lifting device, and a rocking handle. The hopper is arranged to correspond to the discharge port. and the hopper is connected to the lifting rope, and the lifting rope is installed on the lifting device; and the lifting device is drivingly connected to the rocking handle to drive the hopper to move up and down through the lifting rope, thereby completing material discharging.

In the embodiment, the in-site compositing system for community kitchen waste further includes a solar power generation unit and a monitoring unit, where the solar power generation unit is arranged on a rain shelter and electrically connected to the energy storage unit: the monitoring unit is electrically connected to the energy storage unit, and the monitoring unit is configured to monitor an operation condition of the biological conversion unit in real time and control feeding and discharging rates. a stirring rate and a ventilation rate of the biological conversion unit.

In the embodiment, the energy storage unit includes a transformer and an energy storage battery, wherein the transformer is electrically connected to the generator, and the transformer is electrically connected to the energy storage battery: the energy storage battery is electrically connected to the monitoring unit and the biological conversion unit, and the energy storage battery is configured to provide the electrical energy to the monitoring unit and the biological conversion unit.

Beneficial effects are as follows:

A community composting system in-site for kitchen waste according to embodiments of the present disclosure combines organically a kitchen waste bioresource system with a collection system to solve the transportation and land occupation problems caused by the separation of collection and disposal in the past: and the design of the roller fermentation bin driven by both power and electricity solves the problem of energy supply for in-site processing and operation of kitchen waste, and can effectively reduce the disposal costs.

### BRIEF DESCRIPTION OF THE DRAWINGS

FIG. 1 is a schematic diagram of an overall structure of a community composting system in-site for kitchen waste according to embodiments of the present disclosure.

1. solar panel: 2. gravity sensing gate: 3. crushing and removing impurity device: 4. front-end sealing bin: 5. roller fermentation bin: 6. stirring device: 7. anti-sideslip pulley set: 8. supporting seat: 9. monitoring unit: 10. induced draft fan: 11. energy storage unit: 12. electric motor: 13. gear transmission mechanism: 14. discharge port: 15. hopper: 16. speed change device; 17. driveshaft set: 18. rear-end sealing bin: 19. air outlet pipe: 20. barrel discharge port: 21. deodorization filter element: 22. barrel limiter: 23. rocking handle; 24. control panel: 25. lifting device: 26. discharge barrel; 27. fitness equipment; 28. rain proof hood.

### DETAILED DESCRIPTION OF THE EMBODIMENTS

### Embodiment 1

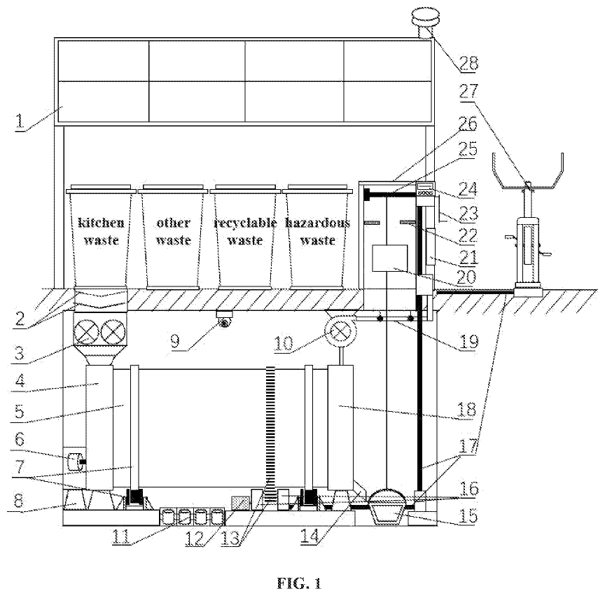

Referring to FIG. 1, an in-site composting system for community kitchen waste. including:

a dump unit, configured to receive a kitchen waste:

a power energy conversion unit, configured to convert a part of energy generated by a fitness equipment 27 or a rocking handle 23 into electrical energy, and output a remaining part of the generated energy as direct power;

a biological conversion unit, configured to cooperate with the dump unit and receive the power output by the power energy conversion unit, where the biological conversion unit is configured to receive the kitchen waste fed by the dump unit and perform a composting and fermentation processing on the kitchen waste;

an energy storage unit 11, connected to the power energy conversion unit and a solar panel 1. and configured to store the electrical energy generated by the power energy conversion unit and the solar panel 1: and

a material outputting and returning unit. connected to the biological conversion unit, and configured to output and return a material decomposed by the biological conversion unit

In this embodiment, the dump unit and the power energy conversion unit are located on the ground, and the biological conversion unit is located underground.

In this embodiment, the power energy conversion unit includes the fitness equipment 27, a transmission assembly, and a power generation assembly. The transmission assembly includes a flywheel assembly and a driveshaft set 17, where a main rotating wheel of the fitness equipment 27 is connected to the fly wheel assembly in a transmission manner through a directly connected transmission shaft, and the driveshaft set 17 includes a first transmission shaft, a second transmission shaft, and a third transmission shaft.

The flywheel assembly is connected to a first end of the first transmission shaft, and the first transmission shaft and the second transmission shaft are arranged vertically. A second end of the first transmission shaft is drivingly connected to a non-end part of the second transmission shaft through a transmission component, where the transmission component may be a worm and worm wheel mechanism. a worm is installed at the second end of the first transmission shaft, and a worm wheal is installed at the non-end part of the second transmission shaft, and the worm wheel and the worm cooperate to achieve transmission.

A first end of the second transmission shaft is provided with a transmission device, the transmission device is drivingly connected to a first end of the third transmission shaft, and the first end of the third transmission shaft is engaged to an input end of the power generation assembly.

In this embodiment, the transmission device is a gearbox: the power generation assembly includes a generator and a current collecting device. and an input end of the gearbox is drivingly connected to the first end of the second transmission shaft: an output end of the gearbox is drivingly connected to the first end of the third transmission shaft, and a second end of the third transmission shaft is drivingly connected to the generator through a coupling; and a brake is arranged on the gearbox: and the current collecting device is configured to collect the electrical energy generated by the generator and transmit the electrical energy to the energy storage unit 11.

In this embodiment. the dump unit includes a kitchen waste can, a gravity sensing gate 2. a crushing and removing impurity device 3, and a feeding pipeline. The gravity sensing gate 2 is arranged at a bottom of the kitchen waste can. The crushing and removing impurity device 3 is arranged at a lower end of the gravity sensing gate 2. and is configured to remove impurities in the kitchen waste and then crush the kitchen waste.

The feeding pipeline is arranged at a lower end of the crushing and removing impurity device 3, and is configured to transport the kitchen waste processed by the crushing and removing impurity device 3 to the biological conversion unit.

In this embodiment, the gravity sensing gate 2 is located at the bottom of the kitchen waste can. When waste in the kitchen waste can reaches a preset value, the gravity sensing gate 2 opens automatically.

In this embodiment, the crushing and removing impurity device 3 includes a receiving box, a bag breaking and impurity removing needle roller, a cleaning mechanism, and a screen mesh. The receiving box is arranged to correspond to the gravity sensing gate 2, and is used to receive the kitchen waste released by the gravity sensing gate 2.

There are two bag breaking and impurity removing needle rollers, and the two bag breaking and impurity removing needle rollers may be rotatably arranged in the receiving box, where the two bag breaking and impurity removing needle rollers rotate in opposite directions: and the bag breaking and impurity removing needle rollers are provided with inverted hook-shaped steel needles, and the inverted hook-shaped steel needles are used to sort out the extruded plastic bags.

The screen mesh is arranged at the lower end of the two bag breaking and impurity removing needle rollers, and is used to receive and screen out the plastic and the crushed kitchen waste.

The cleaning mechanism is arranged at one side of the screen mesh and is used to collect the screened objects on the screen mesh into a collection bucket. In this embodiment, the cleaning mechanism may be a scraper that can move relative to the screen mesh, or may be a component that can clean up the screened objects on the screen mesh.

The feeding pipeline is arranged at the lower end of the screen mesh and is used to transport kitchen waste that passes through the screen mesh to the biological conversion unit.

In this embodiment, the biological conversion unit includes a roller fermentation bin 5, a micro-negative-pressure deodorization device, a gear transmission mechanism, a stirring device 6, and a driving device. The roller fermentation bin 5 may be rotatably installed on a base.

The front and rear ends of the roller fermentation bin 5 are respectively provided with a front-end sealing bin 4 and a rear-end sealing bin 18.

In this embodiment, a supporting seat 8 is arranged at the lower end of the front-end sealing bin 4 and the rear-end sealing bin 18, and the supporting seat 8 is fixedly installed on the base

The anti-sideslip pulley sets 7 are arranged near the end parts of the front and rear ends of the roller fermentation bin 5, respectively.

The micro-negative pressure deodorization device includes a breather pipe, an induced draft fan 10, an air outlet pipe 19. and a deodorization filter element 21. A first end of the breather pipe is communicated with a top of the rear-end sealing bin 18, and a second end of the breather pipe is connected to an inlet end of the induced draft fan 10; an outlet end of the induced draft fan 10 is connected to a first end of the air outlet pipe 19, and the deodorization filter element 21 is arranged on the air outlet pipe 19 and located at a non-end position of the air outlet pipe 19; and an end part of a second end of the air outlet pipe 19 is provided with a rain proof hood 28.

In this embodiment, the odorous air generated in the roller fermentation bin 5 enters the deodorization filter element 21 (biological filter material) through the breather pipe and air outlet pipe 19 under the action of negative pressure and is deodorized and then discharged into the atmosphere. The biological filter material is located at the upper end, positioned on the ground, of air outlet pipe 19. and may be replaced at any time.

The gear transmission mechanism 13 includes an outer ring gear arranged on an outer wall of the roller fermentation bin 5 and a first gear arranged on a fourth transmission shaft, where the first gear meshes with the outer ring gear.

The stirring device 6 includes a stirring shaft and a blade, where the stirring shaft is located inside the roller fermentation bin 5 and is sealed and rotatably connected to the roller fermentation bin 5; a quantity of the blades is provided, and the plurality of the blades are arranged on a circumferential outer side of the stirring shaft.

The driving device includes an electric motor 12 arranged on an outer side of the roller fermentation bin 5. and an output shaft of the electric motor 12 is drivingly connected to the fourth transmission shaft, where a speed change device 16 is provided between the output end of electric motor 12 and the fourth transmission shaft.

A feeding port of the front-end sealing bin 4 is arranged to correspond to the feeding pipeline, and a bottom of the rear-end sealing bin 18 is provided with a discharge port 14.

In this embodiment, the material outputting and returning unit includes a hopper 15, a lifting rope. a lifting device 25, and a rocking handle 23. The hopper 15 is arranged to correspond to the discharge port 14, the hopper 15 is connected to the lifting rope. and the lifting rope is installed on the lifting device 25: and the lifting device 25 is drivingly connected to the rocking handle 23 to drive the hopper 15 to move up and down through the lifting rope, thereby completing material discharging.

The in-site composting system for community kitchen waste according to this embodiment further includes a solar power generation unit and a monitoring unit 9, where the solar power generation unit is arranged on a rain shelter and electrically connected to the energy storage unit 11: the monitoring unit 9 is electrically connected to the energy storage unit 11, and the monitoring unit 9 is configured to monitor an operation condition of the biological conversion unit in real time, and control feeding and discharging rates, a stirring rate and a ventilation rate of the biological conversion unit.

In this embodiment, the energy storage unit 11 includes a transformer and an energy storage battery, where the transformer is electrically connected to the generator, and the transformer is electrically connected to the energy storage battery: the energy storage battery is electrically connected to the monitoring unit 9 and the biological conversion unit, and the energy storage battery is configured to provide the electrical energy to the monitoring unit 9 and the biological conversion unit.

In this embodiment, a discharge barrel 26 is also provided in the rain shelter, and the discharge barrel 26 is provided with a barrel discharge port 20: the discharge barrel 26 is provided with a barrel limiter 22 at the upper end of the barrel discharge port 20. for limiting the discharge position of the hopper 15 where a control panel 24 is arranged at the upper end of the discharge barrel 26, and the control panel 24 is electrically connected to the lifting device 25, the electric motor 12, and the induced draft fan 10, for starting and stopping the lifting device 25, the electric motor 12, and the induced draft fan 10.

The above are only preferred embodiments according to the present disclosure and does not limit the technical scope of the present disclosure. Therefore, any minor modifications, equivalent changes or modifications made to the above-mentioned embodiments based on the technical essence of the present disclosure still fall within the scope of the technical solution of the present disclosure.

What is claimed is:

1. An in-site composting system for community kitchen waste, comprising:
a dump unit, configured to receive a kitchen waste:
a power energy conversion unit, configured to convert a part of energy generated by a fitness equipment or a rocking handle into electrical energy, and output a remaining part of the generated energy directly as power:
a biological conversion unit, configured to cooperate with the dump unit and receive the power output by the power energy conversion unit. wherein the biological conversion unit is configured to receive the kitchen waste fed by the dump unit and perform a composting and fermentation processing on the kitchen waste:
an energy storage unit, configured to be connected to the power energy conversion unit and a solar panel, wherein the energy storage unit is configured to store the electrical energy generated by the power energy conversion unit and the solar panel; and
a material outputting and returning unit. configured to be connected to the biological conversion unit, wherein the material outputting and returning unit is configured to output and return a material decomposed by the biological conversion unit.

2. The in-site composting system for community kitchen waste according to claim 1. wherein the power energy conversion unit comprises the fitness equipment, a transmission assembly. and a power generation assembly: the transmission assembly comprises a flywheel assembly and a driveshaft set, wherein a main rotating wheel of the fitness equipment is connected to the flywheel assembly in a transmission manner through a directly connected transmission shaft: the driveshaft set comprises a first transmission shaft, a second transmission shaft, and a third transmission shaft:
wherein the flywheel assembly is connected to a first end of the first transmission shaft, and the first transmission shaft and the second transmission shaft are arranged vertically: wherein a second end of the first transmission shaft is drivingly connected to a non-end part of the second transmission shaft through a transmission component; and
a first end of the second transmission shaft is provided with a transmission device, the transmission device is drivingly connected to a first end of the third transmission shaft, and the first end of the third transmission shaft is engaged with an input end of the power generation assembly.

3. The in-site composting system for community kitchen waste according to claim 2, wherein the transmission device is a gearbox: the power generation assembly comprises a generator and a current collecting device, an input end of the gearbox is drivingly connected to the first end of the second transmission shaft; an output end of the gearbox is drivingly connected to the first end of the third transmission shaft. and a second end of the third transmission shaft is drivingly connected to the generator through a coupling; and a brake is arranged on the gearbox: and the current collecting device is configured to collect the electrical energy generated by the generator and transmit the electrical energy to the energy storage unit.

4. The in-site composting system for community kitchen waste according to claim 3, wherein the dump unit comprises a kitchen waste can, a gravity sensing gate. a crushing and removing impurity device, and a feeding pipeline, and the gravity sensing gate is arranged at a bottom of the kitchen waste can; the crushing and removing impurity device is arranged at a lower end of the gravity sensing gate, and is configured to remove impurities in the kitchen waste and then crush the kitchen waste; and
the feeding pipeline is arranged at a lower end of the crushing and removing impurity device, and is configured to transport the kitchen waste processed by the crushing and removing impurity device to the biological conversion unit.

5. The in-site composting system for community kitchen waste according to claim 4, wherein the biological conversion unit comprises a roller fermentation bin, a micro-negative pressure deodorization device, a gear transmission mechanism, a stirring device, and a driving device: the roller fermentation bin is rotatably installed on a base;
front and rear ends of the roller fermentation bin are respectively provided with a front-end sealing bin and a rear-end sealing bin:
the micro-negative pressure deodorization device comprises a breather pipe, an induced draft fan, an air outlet pipe. and a deodorization filter element: a first end of the breather pipe is communicated with a top of the rear-end sealing bin, and a second end of the breather pipe is connected to an inlet end of the induced draft fan; an outlet end of the induced draft fan is connected to a first end of the air outlet pipe, and the deodorization filter element is arranged on the air outlet pipe and located at a non-end position of the air outlet pipe: and an end part of a second end of the air outlet pipe is provided with a rain proof hood:
the gear transmission mechanism comprises an outer ring gear arranged on an outer wall of the roller fermentation bin and a first gear arranged on a fourth transmission shaft, wherein the first gear meshes with the outer ring gear:
the stirring device comprises a stirring shaft and a plurality of blades, wherein the stirring shaft is located inside the roller fermentation bin and is sealed and rotatably connected to the roller fermentation bin: the plurality of the blades are arranged on a circumferential outer side of the stirring shaft:
the driving device comprises an electric motor arranged on an outer side of the roller fermentation bin, and an output shaft of the electric motor is drivingly connected to the fourth transmission shaft: and
a feeding port of the front-end sealing bin is arranged to correspond to the feeding pipeline. and a bottom of the rear-end sealing bin is provided with a discharge port.

6. The in-site composting system for community kitchen waste according to claim 5, wherein the material outputting and returning unit comprises a hopper. a lifting rope, a lifting device, and a rocking handle; the hopper is arranged to correspond to the discharge port, and the hopper is connected to the lifting rope, and the lifting rope is installed on the lifting device; and the lifting device is drivingly connected to the rocking handle to drive the hopper to move up and down through the lifting rope, thereby completing material discharging.

7. The in-site composting system for community kitchen waste according to claim 6, further comprising a solar power generation unit and a monitoring unit, wherein the solar power generation unit is arranged on a rain shelter and electrically connected to the energy storage unit: the monitoring unit is electrically connected to the energy storage unit, and the monitoring unit is configured to monitor an operation condition of the biological conversion unit in real time and control feeding and discharging rates, a stirring rate and a ventilation rate of the biological conversion unit.

8. The in-site composting system for community kitchen waste according to claim 7, wherein
the energy storage unit comprises a transformer and an energy storage battery, wherein the transformer is electrically connected to the generator. and the transformer is electrically connected to the energy storage battery: the energy storage battery is electrically connected to the monitoring unit and the biological conversion unit, and the energy storage battery is configured to provide the electrical energy to the monitoring unit and the biological conversion unit.
